(19) United States
(12) Patent Application Publication
(10) Pub. No.: US 2026/0062167 A1
(43) Pub. Date: Mar. 5, 2026

(54) METHOD FOR THE MANIPULATION OF STERILIZED OBJECTS IN A CONTROLLED ATMOSPHERE CHAMBER, CORRESPONDING DEVICE, CORRESPONDING OBJECTS AND CORRESPONDING MACHINE

(71) Applicant: I.M.A. INDUSTRIA MACCHINE AUTOMATICHE S.P.A., OZZANO DELL'EMILIA (BO) (IT)

(72) Inventor: Gabriele GABUSI, OZZANO DELL'EMILIA (BO) (IT)

(21) Appl. No.: 18/873,431

(22) PCT Filed: Jun. 23, 2023
(86) PCT No.: PCT/IT2023/050150

§ 371 Date: Dec. 10, 2024

(30) Foreign Application Priority Data
Jun. 27, 2022 (IT) IT102022000013441

(51) Int. Cl. B65B 63/00; A61L 2/07; A61L 2/20; A61L 2/26; B25J 21/02; B65B 35/16; B65B 55/02; B65B 55/12

(52) U.S. Cl. CPC B65B 63/00; A61L 2/07; A61L 2/208; A61L 2/26; B65B 35/16; B65B 55/027; B65B 55/12; A61L 2103/05; A61L 2103/15; A61L 2202/122; B25J 21/02

(57) ABSTRACT

The invention concerns a method for the manipulation of sterilized objects (22, 23, 24) in a controlled atmosphere chamber (12), comprising a step of gripping at least one of the sterilized objects (22, 23, 24) from the outside of the chamber (12), and a step of inserting the latter inside the chamber (12) through an access aperture defined in correspondence with at least one wall (15, 16) thereof, subsequently a step of closing the access aperture and a step of decontaminating the interior of the chamber (12) by delivering a decontaminating agent.

### FIELD OF THE INVENTION

The present invention concerns a method for the manipulation of sterilized objects in a controlled atmosphere chamber of a processing unit, a corresponding protection device for protecting objects that can be inserted into said controlled atmosphere chamber, the use of the protection device as above, an object which can be inserted into said controlled atmosphere chamber and a packaging machine which comprises a controlled atmosphere chamber, in particular a machine for packaging sterilized products, or products which are to be packaged in a controlled atmosphere, such as for example medical, pharmaceutical, cosmetic or similar products.

### BACKGROUND OF THE INVENTION

Machines are known for packaging products whose sterility must also be guaranteed during the packaging steps, such as for example medical, pharmaceutical, cosmetic and similar products. These packaging machines have at least one processing unit contained in a controlled atmosphere chamber with hermetically sealed walls, so as to isolate the interior from the external environment.

In order to be able to assemble the objects of the processing unit inside the chamber, its walls, typically configured as glass doors hinged to the machine frame, have specific ports called RTPs (acronym of the English Rapid Transfer Port) or Alpha-Beta ports, which allow objects to be introduced into the chamber without compromising the controlled atmosphere inside it, as well as waterproof gloves anchored to other ports in the walls and which allow a technician stationed outside the chamber to manipulate the components inside the chamber while keeping it hermetically closed.

Alpha-Beta ports comprise a primary closing unit, also referred to as an “Alpha unit”, for example consisting of a flange that surrounds an access aperture that can be closed by a door, and suitable to couple with a mating secondary closing unit, also referred to as a “Beta unit”, provided on a transfer device, for example a cylindrical transport container, by means of suitable reciprocal coupling interfaces.

The cylindrical transport container is configured to transport inside and outside the chamber one or more of said objects, configured as modular elements, which have to be sterilized before being introduced into the chamber. In the state of the art, the step of sterilizing these elements takes place in an autoclave while they are already inside the cylindrical transport container. In practice, it is provided to dispose in the autoclave the cylindrical transport container already filled with the modular elements to be sterilized, before the insertion into the chamber. Objects too large to pass through the Alpha-Beta ports are also sterilized in the autoclave before being assembled in the controlled atmosphere chamber.

Alpha-Beta ports have a limited diameter, typically up to 350 mm, and therefore do not allow to insert objects that are of such sizes that they cannot pass through the passage aperture. These objects which do not pass through the Alpha-Beta ports are usually supplied covered by a covering element and enclosed in a closed bag. The covering element and the bag are made of filter material, for example filter paper.

To carry out the assembly it is necessary to open an access aperture made on a wall of the chamber, thus compromising the protected atmosphere. An operator, or possibly two in the case of very heavy and bulky objects, then inserts the object inside the chamber, placing it in the intended position.

A first embodiment of the methods known in the state of the art provides, at this point, to close the access aperture so as to again isolate the atmosphere inside the chamber with respect to the outside. A decontamination cycle is carried out with the decontaminating agent, typically using vaporized hydrogen peroxide, known by the acronym VHP (Vapor Hydrogen Peroxide), at the end of which the operator stationed outside the chamber, using the waterproof gloves anchored to its walls, opens the bag with the aid of scissors or other sharp instruments, removes it and subsequently assembles the object in the machine.

We must clarify that this decontamination step must be distinguished from the previous step of sterilization in the autoclave.

The decontamination cycle is performed once the assembly operations of the object in the chamber have been completed. Decontamination allows to confer on the chamber the necessary hygiene and cleaning requirements, since the chamber as a whole cannot be sterilized in an autoclave. As we said, only some objects that come into contact, directly or indirectly, with the product to be packaged have previously been sterilized in an autoclave, while the parts of the machine that will not come into contact, directly or indirectly, with the product to be packaged need not be sterilized.

One problem with this embodiment is that there are many delicate operations that the operator has to carry out using the gloves anchored to the walls. It is easy to understand how such operations are all the more inconvenient the more the object to be manipulated is disposed in a distant or angled position with respect to the gloves, and/or the more the object to be manipulated is bulky and/or heavy. These characteristics of the object can also make it difficult to remove the bag using the gloves.

Another problem of this embodiment is that during the operations of fastening the objects, which are carried out from the outside using the gloves, the latter could get punctured or break, which would compromise the sterility of the chamber. This is a very problematic circumstance, since replacing the gloves is long and laborious, as it is also necessary to perform a new decontamination cycle and verify that the atmosphere in the chamber is decontaminated again.

A second embodiment of the methods known in the state of the art provides that the operator, after inserting the object into the chamber, cuts the bag in correspondence with the attachment portion of the object itself to the machine, leaving it in any case positioned on the object to be protected, although it is no longer hermetically sealed. The object is then fastened in its position of use and subsequently the operator leaves the chamber, which is closed again in order to isolate the atmosphere inside. At this point the decontamination cycle is carried out with the decontaminating agent and subsequently the operator, using the gloves anchored to the walls of the chamber, removes the bag and extracts it from the chamber.

One problem with this embodiment is that the bag is opened while the access aperture to the chamber is still open, and the operator is present in the chamber so as to carry out the above operations, thus compromising the sterility of the objects which had previously been sterilized in an autoclave.

One problem with known methods is that the material from which the bags are made allows the passage of the decontaminating agent, which is deposited on parts of the object which are intended to come into contact, directly or indirectly, with the products to be packaged. This leads to a potential contamination of the products which is sometimes not acceptable from a regulatory point of view, and in some cases can cause the deterioration of the products themselves.

In another technical field, more precisely that of checking letters or parcels in transit in a postal system for delivering objects, patent application US 2009/047173 A1 discloses a method which provides to protect the inside of a container, including its contents, by means of a rigid waterproof lid that is mating in shape with said container, when it is inserted into a controlled atmosphere chamber in which a method of decontamination based on ethylene oxide or hydrogen peroxide is carried out to eliminate possible contaminants of biological origin, which may be pathogenic and therefore harmful to human health. Since this is a different technical field compared with the known solutions described above, it is obvious that the sizes and shapes of the container are different. Moreover, this document does not even address and solve the problems that concern the methods described above, given that the container does not have to be assembled in the processing chamber and that no step of packaging sterile products is provided, where contact of the sterile products with the agent used in the decontamination method must be avoided.

There is therefore a need to perfect a protection device for protecting objects to be inserted into a controlled atmosphere chamber, the objects themselves, a method for the manipulation of them, as well as a machine for packaging sterile products, which can overcome at least one of the disadvantages of the state of the art.

To do this, it is necessary to solve the technical problem of maintaining the sterility of the parts of the object to be inserted which are intended to come into direct or indirect contact with the products to be packaged, and of preventing the vaporized hydrogen peroxide from depositing on the object to be inserted, and in general any decontaminating agent used in the decontamination cycle inside the chamber after the latter has been opened to insert into it said large objects that do not pass through the Alpha-Beta ports.

In particular, one purpose of the present invention is to provide a protection device which allows to maintain the sterility of the object, or at least of a part of the object in direct or indirect contact with the products to be packaged, both during its transport between the autoclave and the controlled atmosphere chamber, as well as subsequently during the machine set-up operations described above.

Another purpose of the present invention is to provide a protection device which allows to protect the object from decontaminating substances, while still allowing it to be sterilized in an autoclave.

Another purpose of the present invention is to perfect a method for the manipulation of sterilized objects in a controlled atmosphere chamber which allows to maintain the sterility of the objects.

Another purpose of the present invention is to perfect a manipulation method which allows to limit as much as possible the use of gloves anchored to the walls of the controlled atmosphere chamber, in particular, avoiding the need for the operator to carry out operations with gloves which put their integrity at risk.

Another purpose of the present invention is to provide an object that can be inserted into a controlled atmosphere chamber which is configured to be protected by the protection device as above.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes and to resolve the technical problem disclosed above in a new and original way, also achieving considerable advantages compared to the state of the prior art, a method according to the present invention has been embodied for the manipulation of sterilized objects which are subsequently introduced into a controlled atmosphere chamber, wherein the chamber is defined by a plurality of walls. The method comprises a step of gripping at least one of the sterilized objects from the outside of the chamber, a step of inserting the at least one sterilized object into the chamber through an access aperture defined in correspondence with one of the walls, subsequently a step of closing the access aperture in order to restore the seal of the chamber, and a step of decontaminating the interior of the chamber by delivering a decontaminating agent.

In accordance with one aspect of the present invention, before the insertion step, there is provided a step of protecting at least one object in order to protect it in a sealed manner, at least partly, by means of a protection device which comprises a protection member made of a flexible, elastically deformable plastic material impermeable to the decontaminating agent, and having a shape mating with at least one part of the at least one object. In the protection step, the protection member is associated at least with the part of the at least one object, in such a way that at least the part of the at least one object is covered by the protection member so as to prevent the decontaminating agent from coming into contact with the part of the at least one object protected in a sealed manner during the decontamination step.

Doing so achieves at least the advantage of having a protection member which covers the object in a sealed manner, and which does not let the decontaminating agent pass, in particular the vaporized hydrogen peroxide which is usually used to restore the hygiene conditions of the controlled atmosphere chamber after it has been opened to allow the insertion of the object or objects that has/have been sterilized outside the chamber. This significantly limits the risk of contaminating the parts of the object that are intended to come into contact, directly or indirectly, with the product to be packaged, which at the same time allows to preserve its sterility. Another advantage, derived from the impermeability to the decontaminating agent of the material which the protection element is made of, and from the fact that the latter encloses in a sealed manner the part of the object that comes into contact with the products, is that the use of the bag which, in the state of the art, encloses the object, and of the covering member made of filter paper is rendered superfluous. The absence of the bag allows to simplify the operations of manipulating the object inside the controlled atmosphere chamber.

The protection device has been mainly designed to protect objects that do not pass through the access aperture of Alpha-Beta ports. However, it has been observed that this protection device can also be used in a very advantageous manner on objects which, by virtue of their small sizes, can pass through Alpha-Beta ports. In fact, with the protection device as above, the opening of the controlled atmosphere chamber no longer involves the loss of sterility of the parts in direct or indirect contact with the product to be packaged, and allows to prevent—in the subsequent step of decontamination for restoring the conditions of the protected atmosphere—the objects inside the chamber from becoming contaminated with the decontaminating agent. Moreover, thanks to the protection device, the operator can fasten the objects in the position of use while he/she is inside the chamber, when the latter is open, without having to resort to gloves anchored in the walls of the controlled atmosphere chamber. These gloves will only be used to remove the protection device after the chamber has been closed and the decontamination cycle performed. This operation does not jeopardize the integrity of the gloves since both these as well as the protection device are made of soft, non-cutting or blunt material.

Doing so also facilitates the insertion of objects that pass through RTP ports, and also limits the risk of breaking the gloves.

The object to be at least partly protected with the protection device is advantageously a component which, during use, has to be assembled in the chamber, more advantageously a mechanical component, and can have at least an overall dimension greater than 350 mm, which is the maximum diameter of known Alpha-Beta ports. However, as stated, the object can also have a maximum overall dimension that is less than 350 mm.

Preferably, between the step of inserting the at least one of the objects inside the chamber and the subsequent step of closing the access aperture, there is provided a step of assembling the at least one of the objects in the chamber.

In accordance with some embodiments, during the protection step, a filter is associated with the protection member or with the at least one object and is configured to allow the passage of steam during the previous step of sterilization in an autoclave, and at the same time to block the decontaminating agent during the decontamination cycle.

According to one variant, the filter is configured to equalize the pressure present inside the object with the pressure outside it, for example with the pressure inside the controlled atmosphere chamber, or with the pressure present in the autoclave during the sterilization process. In this case, the filter may not have any function correlated to the sterilization step.

In accordance with some embodiments, the protection step provides that the object to be assembled comprises a central body around which there is disposed a rigid ring, preferably in relief, that is, protruding toward the outside, and configured to act as an abutment to the protection member when it is associated with the central body, so that the latter is applied on the object in a sealed manner.

According to some embodiments described here, after the decontamination step, there are provided a removal step in which the protection member is removed from the at least one object, and a subsequent extraction step in which the removed protection member is extracted from the chamber.

In accordance with some embodiments, after the protection step and before the insertion step, a sterilization step is provided in which the object which has been protected in a sealed manner, at least partly, by means of the protection device, is sterilized inside a sterilization chamber, for example an autoclave suitable for this purpose.

In accordance with some embodiments, the object protected in a sealed manner is configured as a modular element.

In accordance with another aspect of the present invention, there is provided a protection device for at least partly protecting, in a sealed manner, a sterilized object which can be inserted into a controlled atmosphere chamber, wherein the protection device comprises a protection member configured to protect at least one part of the object in a sealed manner. The protection member is made of a flexible material, it is impermeable to a decontaminating agent, it is elastically deformable and has a shape mating with the at least one part of the object. The protection member is also configured to be associated at least with the at least one part of the object, in such a way that at least the part of the at least one object is covered by the protection member in order to prevent the decontaminating agent from coming into contact with the part of the object protected in a sealed manner.

Advantageously, the material that the covering member is made of is a plastic material. This material can be selected, for example, from silicone, Ethylene-Propylene Diene Monomer (EPDM) rubber or polyurethane plastic materials. We must clarify that these materials are suitable to guarantee both that the object protected by the covering member is effectively sterilized during the sterilization method in the autoclave, and also that it is correctly protected during the decontamination operations inside the chamber.

Preferably, the protection device comprises a filter associated with the protection member or with the at least one object and configured to allow the passage of steam and to block both the decontaminating agent as well as the entry of microbes, particles and dirt. More preferably, the filter is a hydrophobic filter. Preferably, the filter is of the cartridge filter type and has sizes compatible with those of Alpha-Beta ports. The presence of the filter allows to perform the sterilization of the component in the autoclave before it is inserted in the controlled atmosphere chamber, since the filter allows the sterilizing agent to reach the object to be sterilized.

Please note that the filter can be associated with the covering member or with the object, provided that the sufficient condition that it is placed in fluid communication with a compartment created between the covering member and the protected part of the object is satisfied.

According to some embodiments, the protection member comprises an edge delimiting an aperture and configured to be applied in a sealed manner around the object. In particular, the edge is elastic, in order to create a hermetic seal around the object. The edge is preferably made in a single piece with the protection member. This facilitates the creation of the covering member and eliminates the potential problems of seal between the edge and the rest of the covering member, while guaranteeing the elasticity of the edge.

According to some embodiments, the protection member comprises at least one gripping element made in a single piece with the shaped edge and protruding from it toward the outside with respect to the access aperture. This allows to facilitate the removal of the covering member, which occurs after the controlled atmosphere chamber has been closed and the decontamination process has been carried out to restore the conditions of the protected atmosphere, and therefore this removal is carried out by the operator outside, through the gloves anchored to the walls of the controlled atmosphere chamber.

In order to be able to associate the filter with the protection member, the latter preferably comprises a second sterilization aperture surrounded by an attachment element configured to be associated with the filter, in particular with an attachment portion thereof.

In accordance with one aspect of the present invention, there is provided an object which can be inserted into a controlled atmosphere chamber, in particular of a machine for packaging sterilized products. The object is protected, or intended to be protected, by a protection device as defined above, configured to protect at least one part of the object in a sealed manner. The protection member has a shape mating with at least one part of the object, it is made of a flexible material, it is impermeable to a decontaminating agent and elastically deformable. The protection member is also configured to be associated at least with the at least one part of the object, in such a way that the protection member can cover the at least one part of the object in order to prevent the decontaminating agent from coming into contact with the protected part of the object. For this purpose, the protection member comprises a central body and a rigid ring disposed around the central body, preferably in relief, that is, protruding toward the outside from the central body, and configured to act as an abutment to the protection member in such a way that the latter is applied in a sealed manner on the at least one object.

In the context of the present application, by object we mean an object or component provided to be assembled in the controlled atmosphere chamber. The object can be a mechanical component, for example a modular element able to be assembled on a fixed frame present in the chamber.

In particular, the ring is configured to act as a seating for the insertion of the edge of the protection member, so as to improve the seal around the object and ensure the tightness of the coupling between the object and the protection member.

Preferably, the ring has a circumference with a circular or oval shape. These shapes avoid sharp edges in correspondence with which the tightness of the seal could fail. Other shapes of the ring can be provided, as long as they are suitable to guarantee the hermetic seal.

According to some embodiments, in particular in which the object does not have a cylindrical shape, the ring is misaligned, or eccentric, with respect to the central body of the object. This allows to facilitate the removal of the protection member, placing at least a part of the edge, and potentially also a gripping element integrated in the protection member, at a greater distance from the object.

Preferably, the ring is perpendicular to an axis of development of the central body of the object. Alternatively, the ring can be inclined by an angle of less than 90° with respect to the axis of development of the central body of the object.

The ring can be made in a single piece with the central body of the object, or firmly connected to it, for example by welding or gluing. The ring is configured to define the border between the zone protected by the protection member and the zone not protected thereby, which does not come into contact with the products, and in correspondence with which the attachment means are provided for the fastening in the machine.

Advantageously, the object comprises a second sterilization aperture surrounded by an attachment element configured to be associated with the filter, in particular with an attachment portion thereof.

In accordance with another aspect of the present invention, the use of a protection device as disclosed above is provided, for the manipulation of objects which are already sterilized and subsequently inserted into a controlled atmosphere chamber. In particular, the protection device comprises a covering member having a shape mating with at least one part of the object and it is made of a flexible plastic material, impermeable to a decontaminating agent and elastically deformable. The protection member is also associated at least with the at least one part of the object, in such a way that the protection member can cover the at least one part of the object in order to prevent at least the part of the object from coming into contact with a decontaminating agent.

According to some embodiments of the use, the protection device also comprises a filter which can be associated equally with the protection member or with the at least one object, and configured to allow the passage of steam during the sterilization in the autoclave and at the same time to block the decontaminating agent during the step of decontamination in the chamber.

In accordance with another aspect of the present invention, a machine for packaging products is provided, in particular sterilized products or products to be packaged in a controlled atmosphere, comprising a processing unit provided with a controlled atmosphere chamber which is defined by a plurality of walls, at least one sterilized object which can be inserted into the chamber, wherein the at least one object is at least partly protected by a protection device as disclosed above, that is, a protection device comprising a protection member having a shape mating with at least one part of the object and made of a flexible, elastically deformable material impermeable to a decontaminating agent, so as to prevent the decontaminating agent from coming into contact with the protected part of the at least one object.

According to some embodiments, the protection device also comprises a filter which can be associated equally with the protection member or with the at least one object, and configured to allow the passage of steam during the sterilization in the autoclave and at the same time to block the decontaminating agent during the step of decontamination in the chamber.

Preferably, the component is as disclosed above, that is, it comprises a central body and a rigid ring disposed around the central body and configured to act as an abutment to the protection member in such a way that the latter is applied in a sealed manner on the at least one object.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a partial and schematic three-dimensional view of a packaging machine, comprising a processing unit equipped with a controlled atmosphere chamber;

FIG. 2 is a three-dimensional view of a first object of the packaging machine of FIG. 1, on which a first embodiment of a protection device according to the present invention is assembled;

FIG. 3 is a three-dimensional view of a protection member of the protection device of FIG. 2;

FIG. 4 is a three-dimensional view of the object of FIG. 2, without protection member;

FIG. 5 is a section view of the object of FIG. 2;

FIG. 6 is an enlarged view of the detail A of FIG. 5;

FIG. 7 is a three-dimensional view of a variant of the protection member of FIG. 3, viewed from a different angle;

FIG. 8 is a three-dimensional view of the object of FIG. 2 on which the protection member of FIG. 7 is assembled;

FIG. 9 is a three-dimensional view of a protection member of a second embodiment of the protection device according to the present invention;

FIG. 10 is a three-dimensional view of a second object of the processing unit of FIG. 1;

FIG. 11 is a three-dimensional view of a protection member of a third embodiment of the protection device according to the present invention;

FIG. 12 is a three-dimensional view of a third object of the packaging machine of FIG. 1; and

FIG. 13 is a three-dimensional view of the object of FIG. 12 on which the protection member of FIG. 11 is assembled.

We must clarify that in the present description the phraseology and terminology used, as well as the figures in the attached drawings also as described, have the sole function of better illustrating and explaining the present invention, their function being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

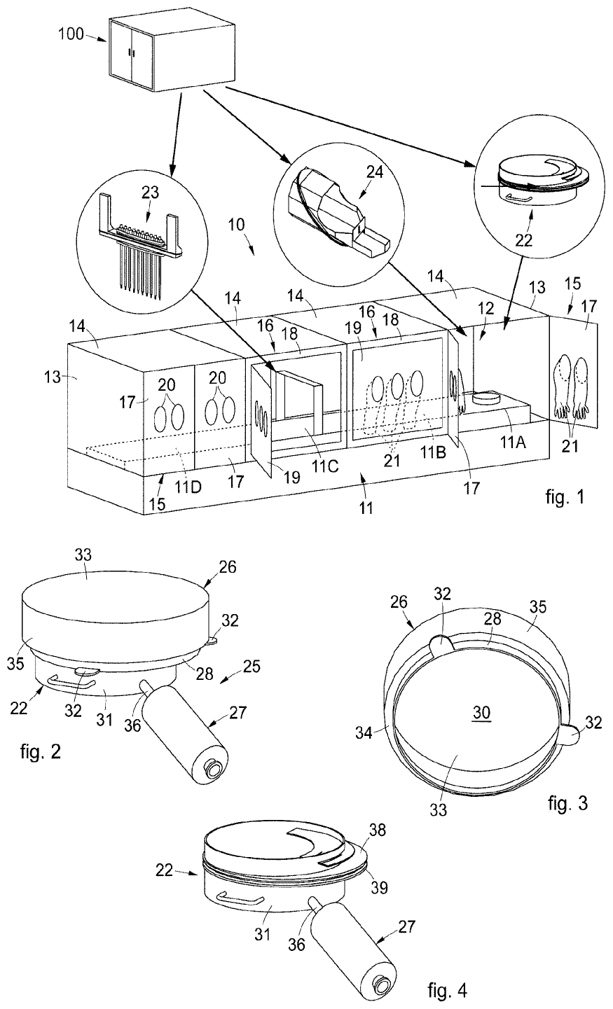

With reference to FIG. 1, there is described a machine 10 for packaging products, in particular sterile products or products to be packaged in a controlled atmosphere such as medical, pharmaceutical, cosmetic products or any other product whose packaging requires operating in a controlled atmosphere. The packaging machine 10 comprises a processing unit 11 provided with a controlled atmosphere chamber 12 of a known type and defined by a plurality of walls 13, 14, 15, 16.

The chamber 12 is defined by fixed lateral walls 13, by fixed upper walls 14 and by walls 15, 16 in correspondence with which an access aperture can be defined for the insertion of the already sterilized objects to be packaged inside the chamber. For this purpose, the walls 13, 14, 15 and 16 of the chamber 12 can be openable. In particular, the chamber 12 comprises a first type 15 of openable wall consisting of two panels 17, for example made of glass, hinged in correspondence with one side thereof, and a second type 16 of openable wall consisting of a frame 18 and a single panel 19, for example made of glass, hinged to it.

The openable walls 15, 16 are each equipped, in a known manner, with one or more portholes 20 to which corresponding manipulation gloves 21 can be anchored or which can form part of an Alpha-Beta port, through which the partial passage of a corresponding cylinder is provided in order to insert objects into the chamber 12 without compromising the controlled atmosphere. The operation of the Alpha-Beta ports is known per se, and will not be described below. The Alpha-Beta port can be of the type marketed by the company Getinge-La Calhène.

In the example shown, the processing unit 11 is divided into four zones 11A, 11B, 11C, 11D, each located in correspondence with a respective openable wall 15, 16 and in which corresponding elements can be inserted and assembled, such as, for example, a vibrating bowl 22, a dispenser 23 and a lid pre-feeder 24. The vibrating bowl 22 and the pre-feeder 24 have to be assembled in the first zone 11A of the processing unit 11, while the dispenser 23 can be assembled in the third zone 11C.

FIG. 2 shows the vibrating bowl 22 on which a protection device 25 is applied, consisting of a protection member 26, commonly referred to as a cover, and a filter 27.

The cover 26 is made in a single piece and of silicone, that is, a plastic material impermeable to a decontaminating agent and elastically deformable. Silicone has the advantage of being easily available and cheap. Other similar materials can be provided, such as EPDM rubber, which has the advantage of not absorbing decontaminating substances such as vaporized hydrogen peroxide, or polyurethane materials.

The cover 26 shown here has been designed to have a shape mating with the upper part of the vibrating bowl 22 so as to be assembled in a sealed manner thereon, in order to cover and isolate at least its interior (FIG. 2). In particular, the cover 26 comprises an edge 28 which delimits a compartment 30 which is internal to the cover itself (FIG. 3). The edge 28 is circular in shape, to better adhere to the central body 31 of the vibrating bowl 22 which has a substantially circular section.

The edge 28, made in a single piece with the cover 26 and therefore also made of silicone, is provided with two gripping tabs 32 to allow a user to grip the cover 26 in order to remove it from the vibrating bowl 22 easily and rapidly.

The cover 26 comprises an upper wall 33 with a circular shape and a lower wall 34 connected to the upper wall by means of a lateral wall 35 and delimited at the lower part by the edge 28.

The filter 27 is a cartridge filter of a known type, in particular with sizes suitable to allow it to pass through Alpha-Beta ports. Specifically, the filter 27 can be of the type marketed by the company Pall Life Sciences under the name Novasip™ filter.

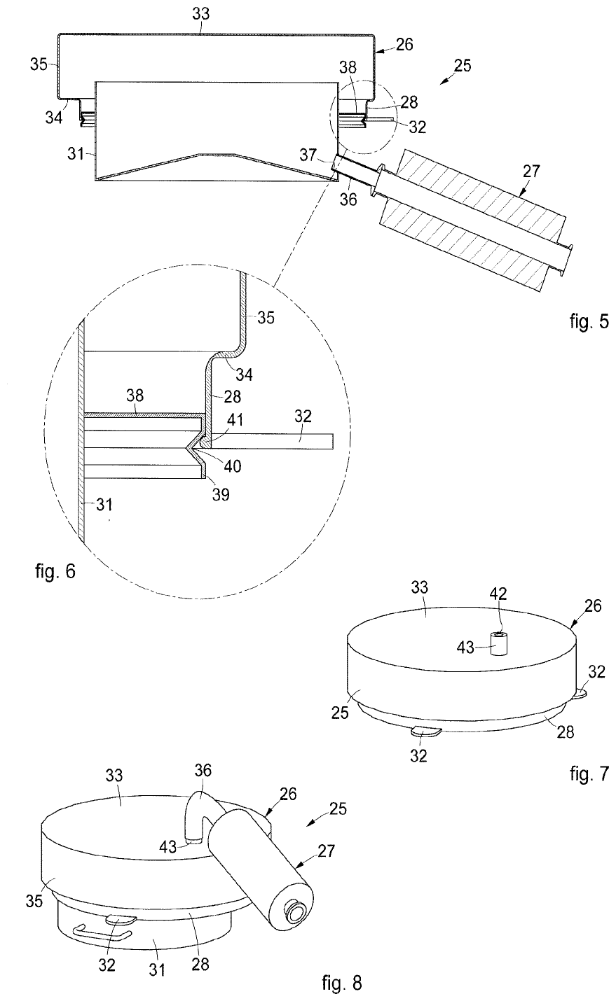

In the example shown, the filter 27 is attached to the vibrating bowl 22 (FIGS. 2, 4 and 5). For this purpose, the filter 27 is equipped with a connection element 36, that is, a cylindrical tube located coaxially to the filter 27, and which connects directly to the vibrating bowl 22 through a sterilization aperture 37 provided therein (FIG. 5) and equipped with known connection means, for example by screwing or shape interference, for simplicity not shown in the drawing.

The vibrating bowl 22 is provided, around a cross section thereof, with a rigid ring 38 made in a single piece therewith and which extends outward and perpendicularly from a lateral wall thereof (FIGS. 5 and 6). The ring 38, in the present example, is contained in a plane perpendicular to an axis of development of the central body 31 of the vibrating bowl 22, in correspondence with an intermediate zone thereof between the upper edge and the bottom.

The ring 38 has a circular-shaped circumference, substantially corresponding to the shape of the edge 28 of the cover 26, so that said edge better adheres to it, to the advantage of a hermetic seal between the cover 26 and the ring 38.

In the example given here, the ring 38 comprises an external wall 39 perpendicular to it and equipped with a circumferential groove 40 which acts as a seating for the edge 28 of the cover 26 (FIGS. 5 and 6). In order to improve the stability and seal in correspondence with the groove 40, the edge 28 advantageously comprises an internal lip 41 which protrudes toward the groove 40 in order to engage with it (FIG. 6).

The ring 38 is preferably misaligned, or eccentric, with respect to the central body 31 of the vibrating bowl 22 so as to have a portion in which the external wall 39 is further away from the external wall of the central body 31 (FIGS. 4 and 5). This allows to facilitate the removal of the cover 26 from the vibrating bowl 22, as will be explained in more detail below.

FIGS. 7 and 8 show a variant of the cover 26 equipped with a sterilization aperture 42 which is equipped with an attachment sleeve 43 to connect the filter 27 thereto. In this case, the vibrating bowl 22 does not necessarily have its sterilization aperture 37, or in any case it is hermetically closed in a known manner, for example by means of a stopper. Since the connection sleeve 43 is provided on the upper wall 33 of the cover 26, here the connection element 36 is provided curved, in such a way as to orient the filter 27 in a more stable position.

The protection device 25 described heretofore allows to maintain the sterility inside the vibrating bowl 22 even after the latter leaves the sterilization chamber, for example an autoclave 100, in which it has been sterilized (FIG. 1), which facilitates its assembly in the processing unit 11 inside the controlled atmosphere chamber 12.

A method for the manipulation of the vibrating bowl provides to dispose the protection device 25 on the vibrating bowl 22, applying the cover 26 thereto, in particular applying the edge 28 of the cover 26 against the external wall 39 of the ring 38, in order to guarantee a hermetic seal with the vibrating bowl 22. Please note that no bag is provided to wrap the assembly consisting of the vibrating bowl 22 and the protection device 25. We must also clarify that the filter 27 of the protection device 25 can be equally fastened to the cover 26 or to the vibrating bowl 22, as described above.

The vibrating bowl 22 equipped with the protection device 25 is then sterilized in a sterilization chamber, for example configured as an autoclave 100 (FIG. 1), the latter is generally autonomous and independent with respect to the chamber 12, since it can also be placed at a considerable distance therefrom, for example even in an industrial plant different from the one in which the chamber 12 is disposed. The sterilization is made possible, despite the impermeable material of the cover 26 and the seal between the latter and the vibrating bowl 22, because of the filter 27, which is configured to let the steam pass during the sterilization in the autoclave and at the same time block the decontaminating agent, that is, the hydrogen peroxide, during the step of decontaminating the chamber 12.

At the end of the sterilization step in the autoclave 100, the vibrating bowl 22 equipped with the protection device 25 is inserted in the processing unit 11 (FIG. 1). Given the sizes of the vibrating bowl 22, it cannot be inserted into the controlled atmosphere chamber 12 through the Alpha-Beta port. It is therefore necessary to introduce the vibrating bowl 22 through the access aperture defined in correspondence with at least one of the walls 13, 14, 15, 16. In the example shown, the access aperture is defined by opening a wall 15 of the chamber 12, in particularly in correspondence with the first zone 11A in the example given here. By way of a non-limiting example, the opening of the wall 15 occurs by rotating the panels 17 which make it up, as shown in FIG. 1. Alternatively, it can be provided that the opening is performed by disassembling a panel of the wall. Obviously, the opening of the wall 15 determines the loss of sterility inside the chamber 12.

Once the wall 15 has been opened, the vibrating bowl 22 is inserted, still equipped with the protection device 25, inside the chamber 12 and it is assembled in the processing unit 11. Please note that the assembly occurs without using the gloves 21 anchored to the walls 15, 16 of chamber 12.

Subsequently, once the assembly is finished, the previously opened wall 15 is closed and a step of decontaminating the inside of the chamber 12 is then carried out by dispensing vaporized hydrogen peroxide, or any other type of suitable decontaminating agent.

Due to the impermeable material of the cover 26 and the presence of the filter 27 which is impermeable to vaporized hydrogen peroxide, the vibrating bowl 22, in particular its internal compartment 30, is protected from any contamination by vaporized hydrogen peroxide, which thus does not deposit on the parts of the vibrating bowl 22 able to come into contact with the products.

Following the decontamination step, the cover 26 is removed. An operator grips one or more gripping tabs 32 through the gloves 21 anchored to the walls 15, 16 of the controlled atmosphere chamber 12, and pulls them in order to widen the edge 28 and thus disengage the latter, in particular its internal lip 41, from the groove 40 of the external wall 39 of the ring 38. The removal of the cover 26 is facilitated by the eccentric position of the ring 38 with respect to the vibrating bowl 22. Indeed, by pulling on a tab 32 located in the portion of the ring 38 furthest away from the central body 31 of the vibrating bowl 22, the edge 28 of the cover 26 is far enough away from the central body 31 to be able to pass over it without having to pull further on the gripping tab 32.

If the filter 27 is connected to the cover 26, it is evident that it is removed together with it. If, on the other hand, the filter 27 is connected to the vibrating bowl 22, it can remain inside the controlled atmosphere chamber 12.

The cover 26 is then extracted from controlled atmosphere chamber 12 through the Alpha-Beta port.

Since the cover 26 of the protection device 25 described heretofore is made of an elastically deformable material, it has a predefined shape which has to mate the object 22, 23, 24 to be protected. The cover 26 described so far has a shape suitable for a vibrating bowl 22.

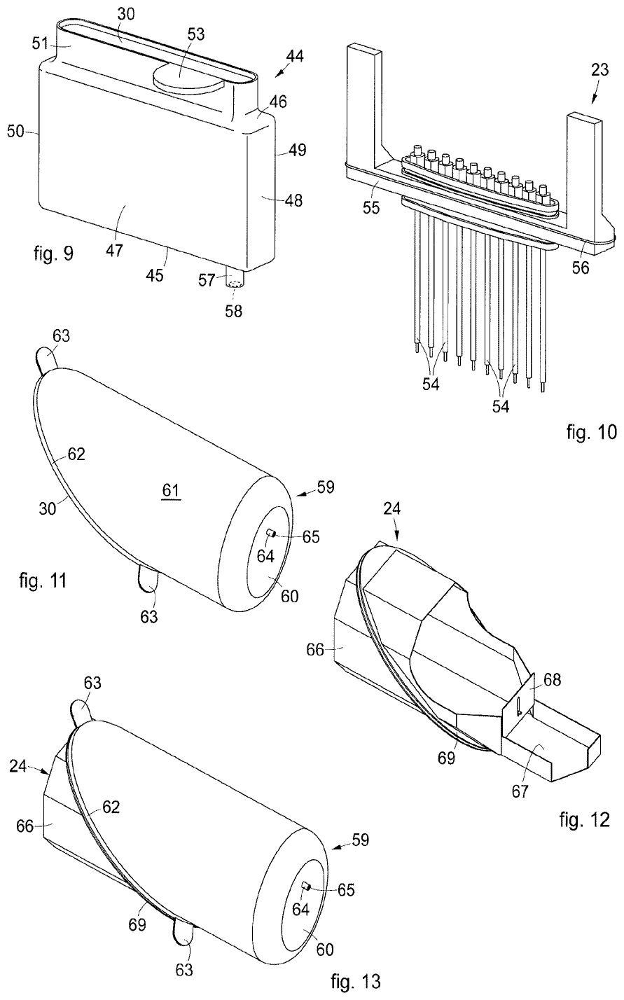

In an alternative embodiment, shown in FIG. 9, a cover 44, provided for the dispenser 23, comprises a lower wall 45, an upper wall 46 and four lateral walls 47, 48, 49, 50 which are shaped to define a parallelepiped compartment, as well as an oblong-shaped edge 51 which delimits an internal compartment 30. Also in this embodiment, the edge 51 is provided with a gripping tab 53.

This cover 44 is suitable to be applied to the dispenser 23 mentioned above, which is equipped with a plurality of aligned nozzles 54 inserted in a support structure 55 (FIG. 10), which acts as a central body for the dispenser 23 and around which there is disposed a ring 56, which is functionally entirely analogous to the ring 38 of the vibrating bowl 22. The ring 56 has a shape mating with the shape of the edge 51 of the cover 44, which in turn is entirely analogous to the edge 28 of the cover 26 (see FIG. 6). The cover 44 is configured to wrap around the nozzles 54 and the edge 51 is configured to create a hermetic seal with the ring 56 of the support structure 55.

In this embodiment, the filter 27 can only be connected to the cover 44, which for this purpose comprises an attachment sleeve 57 which surrounds a sterilization aperture 58.

Please note that the dispenser 23 has lateral sizes which allow it to be inserted in the chamber 12 by means of an Alpha-Beta port, and therefore without compromising the controlled atmosphere. However, it has been observed that the use of the protection device 25 allows to assemble the dispenser 23 without using the gloves 21 anchored to the walls 15, 16 of the controlled atmosphere chamber 12, following the method previously described. Due to the hermetic seal created between the edge 51 of the cover 44 and the ring 56 of the support structure 55, and due to the impermeability of the silicone, the nozzles 54 of the dispenser 23 are well protected from the vaporized hydrogen peroxide used in the step of decontaminating the chamber 12, after the latter is closed once the assembly of the dispenser 23 has been completed.

In another embodiment, shown in FIG. 11, the cover 59 has a shape mating with the pre-feeder 24. For this purpose, the cover 59 has a substantially cylindrical shape and provides a bottom 60 delimiting an internal compartment 30 in cooperation with a lateral wall 61, the latter being delimited at the lower part, that is, on the opposite part with respect to the bottom 60, by an edge 62. Two gripping tabs 63 protrude outward from the edge 62, which are preferably located in a diametrically opposite position with respect to each other. The cover 59 also comprises an attachment sleeve 64 which surrounds a sterilization aperture 65 provided in the bottom 60.

The edge 62 is inclined with respect to an axis of development of the cover 59, by an angle of approximately 45°. The edge 62 therefore has a substantially oval shape and, apart from this shape, has an identical structure to the edges 28, 51 of the covers 26, 44 (shown in FIG. 6).

The pre-feeder 24 (FIGS. 12 and 13), usually provided to feed stoppers, is known per se and has a hollow central body 66, a surface of which is extended to define a feeding ramp 67 open at its free end and partly obstructed by an intermediate wall 68.

The stopper pre-feeder 24 comprises a ring 69 configured to be wrapped in a sealed manner by the edge 62 of the cover 59 (FIG. 13). This ring 68 is advantageously analogous to the rings 28, 56 of the vibrating bowl 22 and of the dispenser 23 (structure shown in FIG. 6), with the exception that it too is inclined by approximately 45° with respect to an axis of development of the central body 66 of the pre-feeder 24, so as to match the edge 62 of the cover 59. The cover 59 is then coaxial with the pre-feeder 24, to the advantage of a smaller overall size of the assembly.

It is clear that modifications and/or additions of parts may be made to the method for the manipulation of objects in a controlled atmosphere chamber, to the protection device 25, to the object able to be inserted in a controlled atmosphere chamber, and to the product packaging machine as described heretofore, without departing from the field and scope of the present invention, as defined by the claims.

It is also clear that, although the present invention has been described with reference to some specific examples, a person of skill in the art will be able to achieve other equivalent forms of method for the manipulation of objects in a controlled atmosphere chamber, of protection device 25, of object able to be inserted in a controlled atmosphere chamber, and of product packaging machine, having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. Method for the manipulation of objects which are already sterilized and subsequently introduced into a controlled atmosphere chamber which is defined by a plurality of walls comprising a step of gripping at least one of said sterilized objects from the outside of said chamber, a step of inserting said at least one sterilized object into said chamber, through an access aperture defined in correspondence with at least one of said plurality of walls, subsequently a step of closing said access aperture and a step of decontaminating the interior of said chamber by delivering a decontaminating agent, wherein, before said insertion step, there is provided a step of protecting at least one of said objects in order to protect it in a sealed manner, at least partly, by means of a protection device which comprises a protection member made of a flexible, elastically deformable plastic material impermeable to said decontaminating agent, and having a shape mating with at least one part of said at least one object wherein in said protection step, said protection member is associated at least with said part of said at least one object, in such a way that at least said part of said at least one object is covered by said protection member so as to prevent said decontaminating agent from coming into contact with the part protected in a sealed manner of said at least one object during the decontamination step.

2. Method as in claim 1, wherein during said protection step, a filter is associated with said protection member or with said at least one object and is configured to allow the passage of steam and to block the passage of said decontaminating agent.

3. Method as in claim 1, wherein said protection step provides that said at least one object comprises a central body around which there is disposed a rigid ring configured to act as an abutment to said protection member when it is associated with said central body.

4. Method as in claim 1, wherein after said decontamination step, there are provided a removal step in which said protection member is removed from said at least one object and a subsequent extraction step in which said protection member is extracted from said chamber.

5. Method as in claim 1, wherein after the protection step and before the insertion step, a sterilization step is provided in which the object which has been protected in a sealed manner, at least partly, by means of said protection device, is sterilized inside a sterilization chamber.

6. Method as in claim 1, wherein said object protected in a sealed manner is configured as a modular element.

7. Protection device for at least partly protecting, in a sealed manner, at least one sterilized object which can be inserted into a controlled atmosphere chamber, said protection device comprising a protection member configured to protect in a sealed manner at least one part of said object, said protection device wherein said protection member is made of a flexible, elastically deformable material impermeable to a decontaminating agent and it has a shape mating with said at least one part of said object, said protection member also being configured to be associated at least with said at least one part of said object, in such a way that at least said part of said at least one object is covered by said protection member in order to prevent said decontaminating agent from coming into contact with the part of said object protected in a sealed manner.

8. Protection device as in claim 7, that wherein it comprises a filter which can be associated with said protection member or with said at least one object and is configured to allow the passage of steam and to block the passage of said decontaminating agent.

9. Protection device as in claim 7, wherein said protection member comprises an elastic edge delimiting an internal compartment and configured to be applied in a hermetically sealed manner around said at least one object.

10. Object which can be inserted into a controlled atmosphere chamber and protected by a protection device comprising a protection member configured to protect in a sealed manner at least one part of said object, wherein said protection member has a shape mating with at least one part of said object and is made of a flexible, elastically deformable material impermeable to a decontaminating agent, said protection member being also configured to be associated at least with said at least one part of said object in such a way that said protection member can cover said at least one part of said object in order to prevent said decontaminating agent from coming into contact with the protected part of said at least one object.

11. Object as in claim 10, wherein said protection member comprises a central body around which there is disposed a rigid ring configured to act as an abutment to said protection member in such a way that the latter is applied in a sealed manner on said at least one object.

12. Use of a protection device for the manipulation of objects which are already sterilized and subsequently inserted into a controlled atmosphere chamber, wherein said protection device comprises a protection member having a shape mating with at least one part of said at least one object and made of a flexible, elastically deformable material impermeable to a decontaminating agent, said protection member being also associated at least with said at least one part of said object in such a way that said protection member can cover said at least one part of said object in order to prevent at least said part of said object from coming into contact with said decontaminating agent.

13. Use as in claim 12, wherein said protection device comprises a filter which can be associated with said protection member or with said at least one object and is configured to allow the passage of steam and to block the passage of said decontaminating agent.

14. Product packaging machine, comprising a processing unit provided with a controlled atmosphere chamber which is defined by a plurality of walls and at least one sterilized object which can be inserted into said chamber, wherein said at least one object is at least partly protected by a protection device comprising a protection member, having a shape mating with at least one part of said at least one object and made of a flexible, elastically deformable material impermeable to a decontaminating agent, so as to prevent said decontaminating agent from coming into contact with the protected part of said at least one object.

15. Packaging machine as in claim 14, wherein said protection device comprises a filter which can be associated with said protection member or with said at least one object and is configured to allow the passage of steam and to block the passage of the decontaminating agent.

16. Packaging machine as in claim 14, wherein said at least one object comprises a central body around which there is disposed a rigid circumferential ring configured to act as an abutment to said protection member in such a way that the latter is applied in a sealed manner on said at least one object.
